(19) United States
(12) Patent Application Publication
(10) Pub. No.: US 2023/0263853 A1
(43) Pub. Date: Aug. 24, 2023

(54) ECTOPARASITICIDE VETERINARY COMPOSITION

(71) Applicant: HAYNE LANE LTD, Devon (GB)

(72) Inventor: James BRINICOMBE, Devon (GB)

(73) Assignee: HAYNE LANE LTD, Devon (US)

(21) Appl. No.: 18/017,133

(22) PCT Filed: Jul. 1, 2021
(86) PCT No.: PCT/GB2021/051671

§ 371 Date: Jan. 20, 2023

(30) Foreign Application Priority Data
Aug. 4, 2020 (GB) 2012083.8

(51) Int. Cl. A61K 36/8962; A61K 9/00; A61K 31/10; A61K 31/198; A61K 36/02; A61P 33/14

(52) U.S. Cl. CPC A61K 36/8962; A61K 9/0056; A61K 31/10; A61K 31/198; A61K 36/02; A61P 33/14

(57) ABSTRACT

The present invention relates to an ectoparasiticide veterinary composition comprising a nutraceutical sourced from Allium sativum comprising at least one compound of the formula (I): wherein n is at least 4 and in which R¹ is selected from the group comprising: hydrogen; halogens; substituted or unsubstituted C₁₋₅ alkyl groups; substituted or unsubstituted phenyl groups; carboxy group; carboalkoxy groups; hydroxymethyl; and trimethylsilylmethyl.

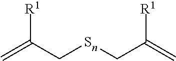

The present invention provides an ectoparasiticide veterinary composition for the treatment, prevention and/or control of ectoparasitic infestations of animals, including terrestrial and/or aquatic animals. In particular, the present invention provides a stable ectoparasiticide veterinary composition comprising diallyl sulphide compositions (DASn), in which n is greater than or equal to 4. Furthermore, the present invention provides an animal feed comprising the ectoparasiticide veterinary composition comprising diallyl sulphide compositions (DASn), in which n is greater than or equal to 4.

### BACKGROUND OF INVENTION

Ectoparasites are organisms which live on the skin of a host animal, from which the ectoparasites derive their sustenance by consuming the host animal's flesh, mucus and/or blood. Many ectoparasites are host specific, while others are not and may be found across a range of host animals. Examples of ectoparasites include, but are not limited to: flukes, leeches, lice, mosquitoes, sand flies, midges, horse flies, deer flies, fleas, ticks and/or mites.

Ectoparasite infestations have been found to have a detrimental effect on the host animal. For example, an ectoparasitic infestation may cause debilitation of the host animal by increasing the risk of exposure of the host animal to bacterial organisms. The ectoparasites may for example create puncture wounds on the host animal which expose the host animal to secondary microorganisms. Furthermore, the ectoparasites may themselves act as vectors for disease transmission to the host animal through feeding.

Large infestations of ectoparasites have been found to debilitate host animals by causing illnesses or diseases associated with one or more of: anaemia, detrimental immune reactions such as for example anaphylaxis or hypersensitivity, dermatitis, low weight gains, irritability, blockage of orifices, inoculation of toxins, tissue damage, or secondary infections.

Ectoparasites affect all species of animal including ruminant, monogastric, insect, terrestrial and water based animals. Examples of animals affected by ectoparasites include: farm animals including cattle, equine, poultry, game, and companion animals such as cats and dogs.

In a farming environment, the animals (such as for example cattle or sheep) can be affected by a range of ectoparasites and nuisance pests. Nuisance flies are an on-going problem for intensive animal holdings. The four fly species found in the largest numbers on worldwide beef cattle units are the house fly, bush fly, stable fly and blowflies. Uncontrolled fly populations may lead to reduced production from flies ‘worrying’ the animals. Flies are also potential carriers of diseases. Ectoparasites can cause a significant economic loss to the farming industry as a result of increased mortality, decreased production, downgrading and potential rejection of animal skins or hides, and reduced reproduction. Common means for controlling flies include insecticidal sprays and/or bait. Repeated use of insecticides can however lead to unwanted residues in the produce and the surrounding environment. Furthermore, repeated use can lead to the development of resistance in flies to the insecticide.

Aquatic animals are also prone to infestations by aquatic ectoparasites. Fish are commonly infested with sea lice which can cause a chronic stress response in the fish resulting in a loss in production and/or resultant meat yield. The likelihood of ectoparasitic infection is higher in confined environments, such as for example in a fish farm, in which the aquatic animals come into close contact with one another.

Existing treatments for ectoparasitic infection of animals, for example aquatic animals, typically include chemical bath treatments (using hydrogen peroxide), drug treatments, heat treatment and mechanical removal of the parasites. There are however a number of disadvantages associated with each of these treatments.

The use of antibiotics and various chemical compounds has resulted in side effects such as drug residues and resistant pathogens in the treated animal. The drug residues may enter the food chain and pose a health risk for human consumers. For example, in the poultry industry, control of red mites (Dermanyssus gallinae) primarily relies on administration of acaricides (such as for example carbamates, amidines, pyrethroids and organophosphates). Due to the repeated use of acaricides, sometimes in high concentrations, red mites may become resistant and acaricides may accumulate in chicken organs, tissues and eggs. It has also been found that strains of ectoparasites have developed which are resistant to the conventional chemical bath treatments and as such the effectiveness of these treatments is reduced. Furthermore, it has been found that it is difficult to accurately control the heat treatment of aquatic animals. The effectiveness of heat treating fish is therefore relatively low. Mechanical removal of ectoparasites is labour intensive and can cause increased stress or harm to the animal which can result in a reduction in feed conversion and growth rates.

In aquatic environments, garlic has been found to promote growth, enhance the immune system, and strengthens the control of pathogens. Garlic contains at least 33 sulphur compounds, 17 amino acids, a number of enzymes and minerals including selenium. One of the most biologically active compounds derived from garlic is allicin (diallyl disulphide). Allicin (2-propene-1-sulfinothioic acid S-2-propenyl ester) is produced by the action of the enzyme alliinase (S-alk(en)yl-L-cysteine sulfoxide lysae) on alliin ((2R)-2-amino-3-[®-prop-2-enylsulfinyl]propanoic acid). Alliin and alliinase are present within separate compartments within the garlic plant and as such the enzymatic conversion only occurs once the garlic bulb is damaged, for example cut or crushed. Once the garlic bulb is cut or crushed, the enzyme alliinase metabolizes alliin to produce allicin. It is however known that allicin is an unstable and biologically active molecule containing two sulphur molecules. Allicin in turn converts to more reduced forms of sulphur such as polysulfides with an allyl functional group attached to either end of the sulphur chain, such as for example diallyl sulphides or DASn, where n is the number of sulphur atoms present in the molecule.

It has been found that diallyl sulphide molecules with short sulphur chain lengths, such as diallyl disulphide, dimethyl disulphide and dimethyl sulphide, are stable and provide a range of biological effects including repellency and insecticidal effects. Polysulfide molecules, in particular diallyl sulfide molecules (DASn) with a higher chain length (i.e. containing three or more sulphur atoms) have also been found to be have increased activity but these molecules are also extremely volatile and highly unstable. The volatility of the higher chain length diallyl sulfide molecules make them unsuitable for use for treatment of ectoparasites.

There is a need for an ectoparasiticide veterinary composition and an animal which effectively reduces or prevents ectoparasitic infestations on a host animal compared to conventional compositions. There is a need for an ectoparasiticide veterinary composition which reduces the attraction between ectoparasites and the host animal, preferably repels the ectoparasites. There is a need for a safe and effective ectoparasiticide veterinary composition which does not have the serious health and environmental problems associated with conventional drug or chemical treatments. There is a need for an all-natural ectoparasiticide veterinary composition. There is also a need for an ectoparasiticide veterinary composition comprising diallyl polysulfides of increased chain length. There is also a need for an ectoparasiticide veterinary composition having improved stability and biological activity. There is a need for an ectoparasticide composition which can be used to reduce and/or prevent ectoparasitic infestations on a host animal with reduced stress experienced by the host animal.

The present invention seeks to address one or more of the above needs.

### SUMMARY OF INVENTION

According to a first aspect, the present invention provides an ectoparasiticide veterinary composition comprising a nutraceutical sourced from Allium, preferably an extract of garlic, Allium sativum, in which the nutraceutical comprises at least one compound of the formula (I):

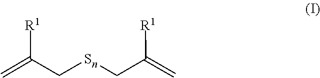

wherein n is at least 4 and in which R¹ is selected from the group comprising:
hydrogen; halogens; substituted or unsubstituted C₁₋₅ alkyl groups; substituted or unsubstituted phenyl groups; carboxy group; carboalkoxy groups; hydroxymethyl; and trimethylsilylmethyl.

The ectoparasiticide veterinary composition of the present invention is effective in the treatment, prevention and/or control of ectoparasitic infestations of animals, including terrestrial and/or aquatic animals, ruminant animals, and/or monogastric animals.

The ectoparasiticide veterinary composition is preferably effective in reducing the attraction of and/or repelling one or more ectoparasites in the vicinity of the target host animal. The ectoparasiticide veterinary composition is preferably effective in the treatment, prevention or control of ectoparasitic infestations caused by all ectoparasites, including for example one or more of: flies, ticks, mosquitos, red mites, Ichthyophthirius multifilis, lice and/or sea lice, or any combination thereof.

In one embodiment, the R¹ is preferably selected from the group comprising: hydrogen; chloro; fluoro; methyl; phenyl; C₂₋₄ alkyl groups; carboxy; carbomethoxy; carboethoxy; hydroxymethyl; and trimethylsilylmethyl.

The compound of formula (I) may also be referred to as diallyl sulfides, or DASn, where n is the number of sulphur atoms within the molecule. The composition of the present invention has improved activity compared to conventional garlic based treatments due to the presence of a stable form of compounds of formula (I) wherein n is at least 4.

Preferably, dially sulphides of the formula DASn, wherein n is 4 or more are present in an amount of at least 5% by weight, preferably at least 10% by weight, more preferably at least 15% by weight, for example at least 20% by weight of the total polysulfides present within the composition Preferably, the composition comprises DASₙ wherein n is at least 5, preferably at least 6.

Preferably, the composition comprises DAS4 in an amount of at least 1%, preferably at least 5%, preferably at least 10%, for example at least 15% by weight. Preferably, the composition comprises DAS4 in an amount of no more than 80%, preferably no more than 60%, preferably no more than 40% by weight. Preferably, the composition comprises DAS4 in an amount of between 1% and 80% by weight, preferably between 5% and 60% by weight, preferably between 10% and 40% by weight, for example between 15% and 40% by weight.

Preferably, the composition comprises DAS5 in an amount of at least 1%, preferably at least 5%, preferably at least 8% by weight. Preferably, the composition comprises DAS5 in an amount of no more than 40%, preferably no more than 30%, preferably no more than 20%, preferably no more than 16% by weight. Preferably, the composition comprises DAS5 in an amount of between 1% and 40% by weight, preferably between 5% and 30% by weight, preferably between 5% and 20% by weight, for example between 8% and 16% by weight.

Preferably, the composition comprises DAS6 in an amount of at least 1%, preferably at least 2%, preferably at least 3%, for example at least 4% by weight. Preferably, the composition comprises DAS6 in an amount of no more than 20%, preferably no more than 15%, preferably no more than 10%, for example no more than 8% by weight. Preferably, the composition comprises DAS6 in an amount of between 1% and 20% by weight, preferably between 2% and 15% by weight, preferably between 3% and 10% by weight, for example between 4% and 8% by weight.

Preferably, the composition comprises DAS7 in an amount of at least 0.5%, preferably at least 1%, preferably at least 2% by weight. Preferably, the composition comprises DAS7 in an amount of no more than 10%, preferably no more than 5%, preferably no more than 4% by weight. Preferably, the composition comprises DAS7 in an amount of between 0.5% and 10% by weight, preferably between 1% and 5% by weight, preferably between 2% and 5% by weight, for example between 2% and 4% by weight.

Preferably, the composition comprises DAS8 in an amount of at least 0.1%, preferably at least 0.5%, preferably at least 1% by weight. Preferably, the composition comprises DAS8 in an amount of no more than 10%, preferably no more than 5%, preferably no more than 2% by weight. Preferably, the composition comprises DAS8 in an amount of between 0.1% and 10% by weight, preferably between 0.5% and 5% by weight, preferably between 1% and 5% by weight, for example between 1% and 2% by weight.

The ectoparasiticide veterinary composition may comprise a plurality of compounds of formula (I). The composition may comprise a plurality of compounds of formula (I) in which n is constant for each of the compounds, or in which n varies between the compounds of formula (I).

At least one, preferably each, compound of the composition has a value of n which is at least 6, preferably at least 7, for example at least 8. The composition may comprise a plurality of compounds of formula (I) in which the value of n varies between the compounds. The composition may comprise a plurality of compounds of formula (I) in which the minimum value of n for the compounds is at least 6, preferably at least 7, for example at least 8.

Preferably, the composition comprises a plurality of compounds of formula (I) in which at least one compound has a value of n which is 6, together with at least one further compound of formula (I) having a value of n which is at least 7. Preferably, the composition comprises a plurality of compounds of formula (I) in which at least one compound has a value of n which is 6, and at least one further compound of formula (I) having a value of n which is at least 7. In one embodiment, the composition comprises a plurality of compounds of formula (I) in which at least one compound has a value of n which is 6, and at least one further compound of formula (I) having a value of n which is 7, optionally together with at least one further compound of formula (I) having a value of n which is at least 8. Preferably, the composition comprises a plurality of compounds of formula (I) in which at least one compound has a value of n which is 7, and at least one further compound of formula (I) having a value of n which is at least 8. In one embodiment, the composition comprises a plurality of compounds of formula (I) in which at least one compound has a value of n which is 7, at least one further compound of formula (I) having a value of n which is 8, optionally together with at least one further compound of formula (I) having a value of n which is greater than 8, such as for example 9.

The ectoparasiticide veterinary composition may comprise at least two compositions of formula (I), for example three of more compositions of formula (I), having n which is greater than 6. Preferably at least one, for example each, of the compositions has a value of n which is greater than 7.

The ectoparasiticide veterinary composition may comprise at least one compound, preferably a plurality of compounds, of the formula (I) in which n is between 6 and 22, preferably between 7 and 22, more preferably between 8 and 22. In one embodiment, the composition may comprise at least 15 compounds of the formula (I), in which each of the 15 compounds has a different n value between 8 and 22. In one embodiment, the composition may comprise at least 22 compounds of the formula (I), in which each of the 22 compounds has a different n value between 1 and 22.

In one embodiment, the ratio of DAS4: DAS5: DAS6: DAS7: DAS8 is approximately 16:8:4:2:1.

The ectoparasiticide veterinary composition may further comprise acetic acid. DAS8 to DAS22 are highly volatile compounds which are sparingly soluble in water. Preferably, the composition comprises acetic acid to solubilise at least one of DAS8 to DAS22. In one embodiment, the acetic acid is present in the form of cider apple vinegar, preferably raw cider apple vinegar with mother.

In one embodiment, the composition comprises water soluble diallyl sulfide compositions of formula (I) and optionally organic soluble diallyl sulfide compositions.

The pH of the ectoparasiticide veterinary composition is preferably within the range of from 4 to 6, preferably between 4.1 to 5.9, such as for example between 4.2 and 4.9.

It has been found that it is preferable for the pH of the composition to be within this range to ensure stability of the compounds of formula (I). When the pH is within this range, it has been found that the composition, and in particular the active compounds of Formula (I), passes successfully, with reduced levels of or essentially without digestion or breakdown, through the digestive system (i.e. through the rumen and stomach and into the small intestine of the host animal). When the pH is within this range, the composition, and in particular the active compounds of Formula (I) can be absorbed into the blood stream at increased levels of uptake thereby increasing the effectiveness of the ectoparasiticide veterinary.

The ectoparasiticide veterinary composition is preferably substantially free from allicin.

The ectoparasiticide veterinary composition is preferably substantially free from dipropyl disulphide (DPDS).

Allicin and dipropyl disulphide are known allergens across certain species of host animals, such as for example equines, canines and felines. Allicin and dipropyl disulphide have been shown to cause haemolytic anaemia and inflammation of the gut. Therefore, the compositions of the present invention have a reduced associated risk of causing an allergic reaction of the host animal, and in particular of causing haemolytic anaemia and inflammation of the gut of the host animal compared to conventional garlic based treatments.

The ectoparasiticide veterinary composition is preferably substantially free from alcohol.

The ectoparasiticide veterinary composition is preferably substantially free from sugar.

The ectoparasiticide veterinary composition may comprise water soluble components and fat soluble components. The compound of formula (I) is preferably a water soluble component which is are able to be absorbed through the bloodstream of the treated animal. The compound of formula (I), comprising diallyl sulphides, has been found to transport the active compounds of formula (I) from digestion so that the active compounds can be transported through the digestive system and absorbed into the vascular system to reach the target site, i.e. the dermis or epidermis, of the host animal without any significant loss in biological activity. The ectoparasiticide veterinary composition may be provided within an “in feed” ectoparasiticide veterinary composition. The term “in feed” is used herein to refer to a composition which is protected from and is therefore immune to the digestive process and can therefore be delivered to a target site on a host animal without any loss or reduction in biological activity. As the composition, in particular compounds of formula (I), of the present invention is distributed through the vascular system, the composition may be effectively distributed throughout the body of the host animal, including the skin and optionally faeces. The ectoparasiticide veterinary compositions of the present invention are delivered effectively over or to the target area, and in some cases over the entire body surface area, i.e. dermis and/or epidermis, of the host animal.

As a result, the ectoparasiticide veterinary compositions of the present invention may provide effective all-body protection to a host animal against ectoparasites.

In one embodiment, the ectoparasiticide veterinary composition further comprises one or more of: allyl methyl disulphide, dimethyl trisulphide, allyl-1-propenyl disulphide, allyl-1-propenyl trisulphide, 2,vinyl-4H-1,3-dithine, methyl-2-propenyl thiosulphinate

In one embodiment, the ectoparasiticide veterinary composition further comprises one or more of:
sulphur-containing precursor compounds for allicin; and/or
an extract of Seaweed; and/or
methionine;
or any combination thereof.

In one embodiment, the sulphur-containing precursor compounds for allicin comprise 2-propenesulphenic acid. It is to be understood that 2-propenesulphenic acid may dimerise, during for example transit through the animal host to form allicin.

The composition may further comprise one or more of: methiin, alliin and/or propiin or any combination thereof.

The composition may further comprise methyl sulfonyl methane (MSM).

The seaweed extract may be an extract of Ascophyllum nodosum. The seaweed extract may comprise one or more amino acids selected from: alanine, aspartic acid, arginine, glutamic acid, glycine, histidine, isoleucine, leucine, lysine, phenylalanine, proline, serine, threonine, tyrosine, and/or valine, or any combination thereof. The seaweed extract preferably comprises phenylalanine.

The seaweed extract may further comprise crude protein and/or minerals such as for example one or more of calcium, iron, potassium, iodine and/or zinc.

The presence of zinc within the ectoparasiticide veterinary composition has been found to have a number of health benefits, such as for example:
to support normal growth (including the development of the skeleton, skin and/or feathers of a host animal);
to enhance the immune system and infectious disease resistance;
to alter the metabolism of carbohydrates, proteins and lipids;
to improve antioxidant abilities;
to influence gene expression by altering DNA and chromatin structure;
to support egg production and development of the egg and shell in poultry;
to support poultry embryo and chick development;
to improve molt performance in poultry.

Preferably, the extract of seaweed improves the transit of the ectoparasiticide veterinary composition through the digestive system of the host animal such that the active compound of Formula(I) is delivered effectively to the target area, such as for example the dermis and/or epidermis, of the animal host. The extract of seaweed preferably sequesters the composition, and in particular the compound(s) of formula (I), of the present invention within and through the digestive system of the animal host.

Methionine is an essential amino acid which is importance for protein synthesis and aiding absorption of amino acids into the intestine of a host animal, as well as being involved in cell proliferation, immune responses and regulation of oxidative stress. Methoinine is however known as a limiting amino acid as it is found in plant sources and cannot be synthesized by a host animal, such as for example by poultry. It is therefore preferably for the ectoparasiticide veterinary composition to include methionine. Supplementation with methionine has been shown to improve amino acid balance and consequently promotes growth performance by enhancing feed efficiency, increasing protein synthesis and decreasing fat synthesis. Methionine preferably migrates to or via the lymph system and skin or epidermis of the host animal. Methionine may alter the smell of the skin of the animal host thereby causing the host animal to be less attractive to parasites. Methionine has been found to alleviate negative effects of heat stress for a host animal.

For example, in one embodiment, the composition may further comprise S-allyl cysteine.

In one embodiment, the composition further comprises an antithrombotic agent, preferably the antithrombotic agent is a naturally occurring antithrombotic agent present within the extract of Allium, such as for example ajoene. The antithrombotic agent may be present within the composition so as to inhibit or prevent the formation of platelets by binding to the fibrinogen receptor.

The composition may further comprise one or more of: chelated zinc, Glycyrrhiza glabra, Urtica dioica, or any combination thereof.

A water extract of Urtica dioica has been found to have powerful antioxidant activity, and can act as a scavenger for free radicals, chelate metals and act as an antimicrobial. Extracts of Urtica dioica have been shown to suppress cytokine production and may have a liver protectant effect

In one embodiment, the composition comprises at least 10% by weight, preferably at least 20% by weight, more preferably at least 30% by weight, for example at least 40% by weight of the at least one compound of formula (I) in relation to the total weight of the composition.

In one embodiment, the composition comprises no more than 90% by weight, preferably no more than 80% by weight, more preferably no more than 70% by weight, for example no more than 60% by weight of the at least one compound of formula (I) in relation to the total weight of the composition.

In one embodiment, the composition comprises between 10% and 90% by weight, preferably between 20% and 80% by weight, more preferably between 30% and 70% by weight, for example between 40% and 60% by weight, of the at least one compound of formula (I) in relation to the total weight of the composition.

In one embodiment, the composition comprises at least 1%, preferably at least 5% by weight, more preferably at least 10% by weight, for example about 14% by weight of sulphur-containing precursor compounds for allicin in relation to the total weight of the composition.

In one embodiment, the composition comprises no more than 50% by weight, preferably no more than 40% by weight, more preferably no more than 30% by weight of sulphur-containing precursor compounds for allicin in relation to the total weight of the composition.

In one embodiment, the composition comprises between 1% and 50% by weight, preferably between 5% and 40% by weight, more preferably between 10% and 30% by weight, for example between 10% and 20% by weight, of sulphur-containing precursor compounds for allicin in relation to the total weight of the composition.

In one embodiment, the composition comprises at least 1%, preferably at least 2% by weight, more preferably at least 5% by weight, for example about 8% by weight of an extract of seaweed in relation to the total weight of the composition.

In one embodiment, the composition comprises no more than 20% by weight, preferably no more than 15% by weight, more preferably no more than 10% by weight of an extract of seaweed in relation to the total weight of the composition.

In one embodiment, the composition comprises between 1% and 20% by weight, preferably between 2% and 15% by weight, more preferably between 5% and 10% by weight, of an extract of seaweed in relation to the total weight of the composition.

According to a second aspect, the present invention provides an animal feed composition comprising an ectoparasiticide veterinary composition as herein described.

The animal feed composition or composition of the present invention is preferably composed entirely of natural ingredients. As a result of the composition or animal feed composition of the present invention being composed entirely of natural materials, it has been found that the composition and animal feed composition may be administered to a host animal without requiring a predetermined withdrawal period prior to meat or egg consumption.

The animal feed composition may be used to feed any suitable target animal. In one embodiment, the animal feed composition is a terrestrial and/or an aquatic animal feed composition.

Suitable target or host animals may include for all animals, for example ruminant animals, monogastric animals, insect animals, terrestrial animals and aquatic animals. Non-exhaustive examples of suitable target or host animals include, but are not limited to, cattle, sheep, goats, horses, chickens, pigs, ducks, birds, dogs, cats and/or fish.

The composition and/or animal feed may be provided in any suitable form such as for example solid, granular or liquid.

The composition and/or animal feed may have an acidic pH.

The animal feed composition may be administered to the target animal using any suitable method of administration. The term “animal feed” is used herein to refer to any means of supplying the composition to an animal, such as for example an animal feed, animal drinking water and/or animal supplements, such as an animal lick or an additive to be added to hay etc. In one embodiment, the animal feed composition is administered in the animal drinking water. As a result, the compositions may be effectively self-administered by the host animal without third party intervention. The compositions and animal feed of the present invention can therefore be administered with reduced handling time and associated stress for the host animal. The compositions and animal feed of the present invention can be administered without requiring any special treatments, yarding, climbing on rails, special applicators or special cleaning techniques. As a result, there are no additional costs or downtime associated with training staff how to administer the compositions or animal feed to the host animal. The compositions and animal feed of the present invention do not require any additional safety equipment such as respirators. The compositions and animal feed of the present invention comprise all natural ingredients and therefore do not require any additional antidotes in case of error or accidental overdosing during administration. Furthermore, as the compositions and animal feed of the present invention comprise all natural ingredients there are no risks associated with accidental contact with the skin of a user. The compositions and animal feed of the present invention may be applied without requiring any additional solvents. The compositions and animal feed of the present invention may be administered with no risk of damage to the coat of an animal host or of causing adverse reactions.

The animal feed composition may comprise any suitable percentage by weight of an ectoparasiticide veterinary composition as herein described depending on the type of animal that is to be treated and the dosage regime for the animal. The animal feed may be administered in any suitable dosage form.

The compositions and animal feed of the present invention can be administered to an animal host to provide effective nutritional and calming support to the animal whilst reducing the attraction, and preferably repelling ectoparasites, to ensure the animal host achieves its optimum genetic potential Advantageously, administration of the compositions and/or animal feed of the present invention does not produce any artificial chemical or drug residue in water, soil, hides or meat of a host animal as a result of treatment of a host animal.

The compositions or animal feed may have an effect on the ectoparasites targeting the host animal almost immediately. The effect of the compositions or animal feed on the animal host may build up over a period of time, from a single dose administration or through repeated does administration, and as such the resultant ectoparasiticide effect of the compositions or animal feed may increase over a period of time to achieve an optimum ectoparasiticide effect, such as over a period of for example 24 hours, 48 hours, 5 days, 1 week, 2 weeks, 1 month, 3 months, or 6 months.

The compositions and animal feed of the present invention do not cause or encourage any form of resistance build up within the target host, i.e. the compositions are resistance-free. As a result, the effectiveness of the ectoparasiticide compositions of the present invention is maintained over repeated administrations.

In one embodiment, the ectoparasiticide compositions and animal feed of the present invention are non-host specific and can therefore be used to treat a range of animal hosts.

In one embodiment, the ectoparasiticide compositions and animal feed of the present invention are non-ectoparasite specific and can therefore be used to treat a range of ectoparasites.

In one embodiment, administration of the compositions and/or animal feed of the present invention enables interruption of the life cycle of parasites, and in some cases the ectoparasite when released from the host animal dies naturally outside of the body of the host animal.

In one embodiment, administration of the compositions and/or animal feed of the present invention results in one or more of the follows:
provision of one or more specific amino acids, such as for example phenylalanine and/or methionine;
provision of nitric oxide in the blood supply of the host animal;
an anti-inflammatory effect on the host animal;
boosting of the immune system of the host animal;
aids balancing of insulin levels in aquatic animals;
encourages healthy benthos in aquatic environments;
support balanced amino acid composition of the diet through the inclusion of methionine;
supporting cell repair through the addition of zinc;
provision of key vitamins and minerals to support animals experiencing a nutritional undersupply;
provision of high levels of fat soluble ingredients to support calmness and comfort, particularly in areas prone to insect attack;
provision of a balanced blend of ingredients to support health and healing;
provision of the appropriate pH to promote palatability.

The main activity areas of the immune system of a host animal are the spleen, the thymus and the marrow, where the increase in white blood cells, antibodies and other enzymes are important for host animal survival. Continual challenges to the immune system, for example to the activity of one or more of the spleen, thymus and/or marrow, of the host animal can weaken the host animal and be detrimental to their prospects for survival. Immune function enhancement places demands on the host animal's energy reserves in order to create cells, antibodies and enzymes. The increased demand on the energy reserves of the host animal can increase the risk of morbidity of the host animal.

According to a further aspect, the present invention provides the use of an ectoparasiticide veterinary composition or an animal feed composition as described herein to improve and/or protect the activity of one or more of: the spleen, thymus and/or marrow of the host animal.

According to a further aspect, the present invention provides the use of an ectoparasiticide veterinary composition or an animal feed composition as described herein to maintain and/or strengthen the performance of the immune system of the host animal.

According to a further aspect, the present invention provides the use of an ectoparasiticide veterinary composition or an animal feed composition as described herein in the treatment, prevention or control of ectoparasitic infestations of host animals.

In one embodiment, the ectoparasiticide veterinary composition or animal feed of the present invention may be used in the treatment, prevention or control of ectoparasitic and endoparasitic infestations of host animals.

In one embodiment, the ectoparasiticide veterinary composition or animal feed of the present invention may be delivered to and act within one or more of: the digestive system, for example the gut; the blood; the organs; and/or the skin of the host animal. Preferably, the ectoparasiticide veterinary composition or animal feed of the present invention may be delivered to and act within one or more of the dermis and/or epidermis of the host animal.

In one embodiment, the ectoparasiticide veterinary composition or animal feed of the present invention may be used in the treatment, prevention or control of ectoparasitic infestations of host animals by reducing the attraction between the ectoparasite(s) and the host animal.

In one embodiment, the ectoparasiticide veterinary composition or animal feed of the present invention may be used in the treatment, prevention or control of ectoparasitic infestations of host animals by causing the ectoparasite(s) to be repelled from the host animal.

In one embodiment, the ectoparasiticide veterinary composition or animal feed of the present invention may be used in the treatment, prevention or control of ectoparasitic infestations of host animals by reducing the attraction between the ectoparasite(s) and the host animal by reducing the blood palatability of the host animal. As a result, ectoparasites are repelled from the host animal. By changing or reducing the blood palatability, the compositions or animal feed of the present invention can reduce or prevent secondary or prolonged biting or feeding by ectoparasites.

In one embodiment, the reduction of blood palatability caused by the ectoparasitic composition or animal feed of the present invention helps the natural release of endoparasites, such as intestinal worms, so that they pass safely out of the host animal in the faeces.

In one embodiment, the ectoparasiticide veterinary composition or animal feed of the present invention may be used in the treatment, prevention or control of ectoparasitic infestations of host animals by reducing the attraction between the ectoparasite(s) and the host animal by reducing or preventing the flow of blood from the host animal to the ectoparasite. Reduction or prevention of blood supply from the host animal to the ectoparasite can reduce the food supply to the ectoparasite thereby stunting the growth of the ectoparasite.

In one embodiment, the ectoparasiticide veterinary composition or animal feed of the present invention may be used in the treatment, prevention or control of ectoparasitic infestations of host animals by reducing the attraction between the ectoparasite(s) and the host animal by altering the odour of the host animal. As a result, the attraction of the ectoparasites to the host animal is reduced, for example the ectoparasites are repelled from the host animal.

In one embodiment, the composition or animal feed of the present invention comprise one or more compounds of Formula (I) which after administration are passed out of the animal host through sweat and into the acid mantle. The compounds of Formula (I) are sulfur containing compounds are antibacterial agents. The antibacterial activity of the sulfur containing compounds alters the breakdown of the sweat and as such alters the close environment of the animal and makes it less attractive or repellent to ectoparasites.

Skin integrity is also an important factor when considering the effect of bites from ectoparasites.

The composition of the present invention is able to travel safely through the digestive system where it is absorbed through the gut wall and into the vascular system. The composition of the present invention then passes to the dermis and epidermis of the host animal. The composition of the present invention, and in particular the compounds of Formula (I), has been found to alter the acid mantle and skin microbiota of the host animal. Parasites detect their host by the expression of volatile organic compounds that are released from the bacteria on the skin of the host animal. Therefore, the administration of the composition of the present invention creates a different “signal” from the host animal to the parasite as a result of the alterations in acid mantle and skin microbiota. The parasite can no longer recognise the host animal as its target host due to this different “signal” and as a result the parasite does not proceed to target or blood feed from the host animal.

In one embodiment, the ectoparasiticide veterinary composition or animal feed of the present invention may be used in the treatment, prevention or control of ectoparasitic infestations of host animals by reducing the attraction between the ectoparasite(s) and the host animal by supporting, maintaining and/or improving the skin integrity of the host animal. The composition and animal feed of the present invention can therefore be used to reduce the ability of an ectoparasite to identify, attack or attach itself to the skin of a host animal. The ectoparasiticide veterinary composition or animal feed of the present invention may be used as one or more, preferably both of, an astringent and/or anti-inflammatory agent thereby helping to reduce scratching and irritation from bites from ectoparasites.

In one embodiment, the ectoparasiticide veterinary composition or animal feed of the present invention may be used in the treatment, prevention or control of ectoparasitic infestations of host animals by reducing the attraction between the ectoparasite(s) and the faeces deposited by the host animal.

In one embodiment, a first portion of the composition or animal feed of the present invention (preferably the compounds of Formula (I)) are transported to a target site on or within the host animal (for example into the dermis and/or epidermis of the host animal), and a second portion of the composition or animal feed (preferably the compounds of Formula (I)) are transported through the gut of the host animal and are eliminated within the faeces. The presence of the composition or animal feed, and preferably the compounds of Formula (I), within the faeces may reduce the attraction, preferably repel additional or the same parasites and/or ectoparasites, from contacting and/or using the deposited faeces as a laying area. For example, the presence of the composition or animal feed, and preferably the compounds of Formula (I), within the faeces may alter the odour of the faeces causing a reduction in attraction of, preferably repulsion of, ectoparasites to the faeces.

The composition and animal feed of the present invention may be used as an antibacterial agent. The compounds of Formula (I) are sulfur containing compounds are antibacterial agents. The antibacterial activity of the sulfur containing compounds may alter the breakdown of the faeces and as such may alter the close environment of the animal faeces and makes it less attractive or repellent to ectoparasites as a laying area.

The composition and animal feed of the present invention may be used to encourage and maintain healthy survival of the Benthos of for an animal holding, such as for example a fish farm.

In one embodiment, the composition of the present invention comprises an additional protein which is transported through the digestive system of the host animal and is deposited within the faeces. This additional protein, once in contact with eggs laid by a parasite/ectoparasite on the faeces causes the outer shell of the egg to thicken preventing the larvae of an ectoparasite, for example a mosquito, within the egg to hatch.

Ectoparasitic infestations may result in the target animal:
producing an increased level of urea; and/or
having increased cortisol levels; and/or
having reduced immunoglobulin levels; and/or
reduced wound healing properties;
compared to healthy animals.

In one embodiment, the ectoparasiticide veterinary composition or animal feed of the present invention may be used in the treatment, prevention or control of increased level of urea production and/or increased glucose utilization in host animals, in particular in host fish.

In one embodiment, the ectoparasiticide veterinary composition or animal feed of the present invention may be used in improving glucose utilisation in host animals.

In one embodiment, the ectoparasiticide veterinary composition or animal feed of the present invention may be used in the treatment, prevention or control of increased cortisol levels in host animals. For example, the ectoparasiticide veterinary composition or animal feed of the present invention may be used in the to reduce cortisol levels in host animals. Cortisol is beneficial to a host animal's health, and in particular host fish health and well being. Cortisol is responsible for:
influencing hydromineral balance;
energy metabolism;
immune function;
growth hormone; and
epithelial cell secretions.

In one embodiment, the ectoparasiticide veterinary composition or animal feed of the present invention may be used in to increase immunoglobulin levels within host animals.

In one embodiment, the ectoparasiticide veterinary composition or animal feed of the present invention may be used in to improve wound healing properties in host animals. For example, the ectoparasiticide veterinary composition or animal feed of the present invention may be used to enhance fibroblasts function in host animals. MSM may be present within the ectoparasiticide veterinary composition or animal feed and may aid wound healing by for example enhancing fibroblasts function. MSM may help to improve and strengthen the skin structure. The composition and animal feed of the present invention may be used as an antiseptic agent.

Water channel proteins (also known as aquaporins) and Claudin tight junction proteins are transmembrane proteins of fish which can be permeated by water molecules. These proteins are fundamental in the osmoregulation and permeability of fish skins, gills, intestine and kidney.

In one embodiment, the ectoparasiticide veterinary composition or animal feed of the present invention may be used in to enhance the function of one or more of: water channel proteins and/or Claudin tight junction proteins of a host fish.

Fish skin expresses a large amount of antimicrobial peptides to protect the fish from bacteria, fungi, virus, algae and parasites. Examples of antimicrobial peptides include hepcidin, beta-defensin like peptides apolipoproteins and piscidin. Fish exposed to stress will cause an increase in synthesis of heat shock proteins (HSPs) also known as stress proteins. These stress proteins, and in particular HSP90 and HSP70, have been found to play an important role in health, in relation to the host response to environmental pollutants, to food toxins and in particular to the development of inflammation, and non-specific immune responses to bacterial or viral infections.

In one embodiment, the ectoparasiticide veterinary composition or animal feed of the present invention may be used to enhance the production of stress proteins, in particular HSP70 and HSP90, within a host animal, in particular within a host fish, to improve immunity and health of the host animal without requiring exposure of the host animal to heat or stress. The present invention can therefore be used in a non-traumatic method of stimulating an enhanced level of stress proteins within a host animal.

In one embodiment, the ectoparasiticide veterinary composition or animal feed of the present invention may be used to enhance the production of nitric oxide within a host animal, in particular within a host fish. This increase in nitric oxide production is considered to be directly linked to the production of stress proteins.

In one embodiment, the ectoparasiticide veterinary composition or animal feed composition may be used in the treatment, prevention or control of stress related behaviour of animals resulting from ectoparasitic infestations.

In one embodiment, the ectoparasiticide veterinary composition of the present invention may be an anti-inflammatory agent.

According to a further aspect of the present invention, there is provided use of an ectoparasiticide veterinary composition or animal feed composition as herein described for the treatment, prevention or control of one or more diseases associated with bacterial infections.

In one embodiment, the ectoparasiticide veterinary composition or animal feed composition may be used in the treatment, prevention or control of one or more diseases associated with one or more of: Escheria coli, Pseudomomnas aeruginosa, Salmonella typhimarium, and/or Staphylococcus aureus.

Preferably, the ectoparasiticide veterinary composition or animal feed composition may be used in the treatment, prevention or control of one or more diseases associated with one or more of: Escheria coli, Pseudomomnas aeruginosa, Salmonella typhimarium.

According to a further aspect of the present invention, there is provided use of an ectoparasiticide veterinary composition or animal feed composition as herein described for the reduction of methane emissions from a host animal.

According to a further aspect of the present invention, there is provided use of an ectoparasiticide veterinary composition or animal feed composition as herein described for the treatment of respiratory diseases associated with the host animal.

Respiratory diseases can be a significant problem when trying to maintain healthy host animals. A wide range of diseases can cause respiratory signs in host animals, such as for example poultry. The most common signs of respiratory diseases include: Sneezing; Open mouthed breathing; Wheezy/gurgling breathing sounds; Ruffled feathers; Discharge around the nostrils and eyes; and/or Head shaking. Respiratory diseases can be caused by for example one or more of: mycoplasma, infectious bronchitis, high dust levels, high ammonia levels, avian rhinotracheitis, infectious laryngotracheitis, and/or avian influenza.

According to a further aspect of the present invention, there is provided use of an ectoparasiticide veterinary composition or animal feed composition as herein described for the treatment of respiratory diseases associated with a bacterial infection of the host animal.

According to a further aspect of the present invention, the ectoparasiticide veterinary composition or animal feed composition is an anti-fungal agent. The composition may be a selective anti-fungal agent exhibiting no activity against live yeast. As such, the composition of the present invention may be administered with pre-biotic and pro-biotics feed diets without any negative effects on the live yeast.

Embodiments of the present invention will now be described in further detail in relation to the accompanying Figures.

### BRIEF DESCRIPTION

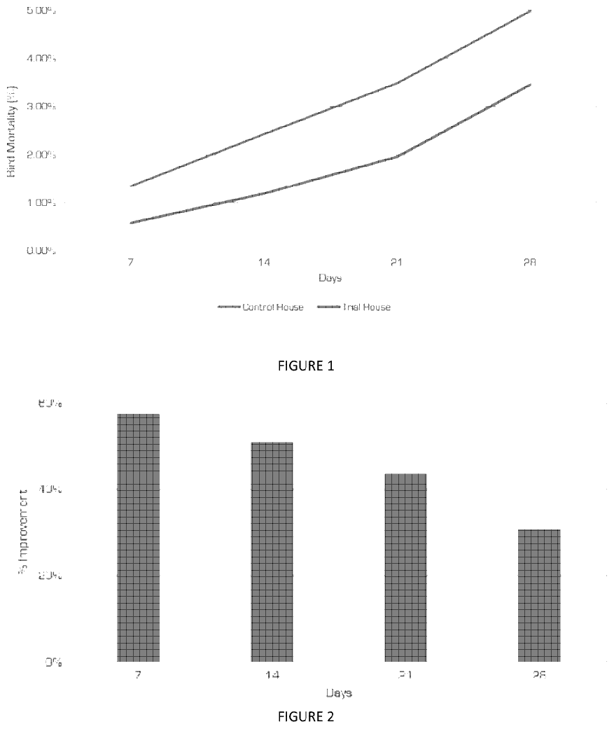

FIG. 1 is a graph illustrating the effect of the ectoparasiticide veterinary composition of the present invention on the mortality of poultry over time;

FIG. 2 is a graph illustrating the improvement in mortality of poultry when treated with the ectoparasiticide veterinary composition of the present invention compared to a control group of poultry;

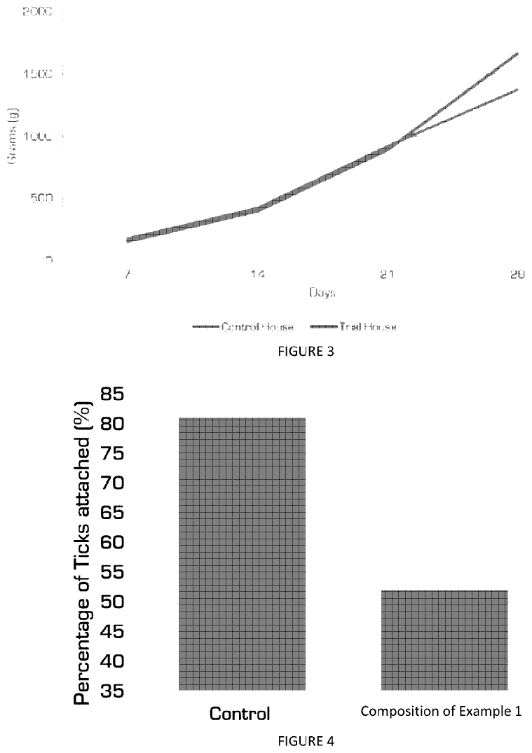

FIG. 3 is a graph illustrating the effect of red mite infestations on the weight of poultry birds;

FIG. 4 is a graph illustrating the effect of the ectoparasiticide veterinary composition of the present invention on the tick infestations on cattle;

FIG. 5 is a graph illustrating the effect of the ectoparasiticide veterinary composition of the present invention on tail swishing of calves during the loading and activity phases;

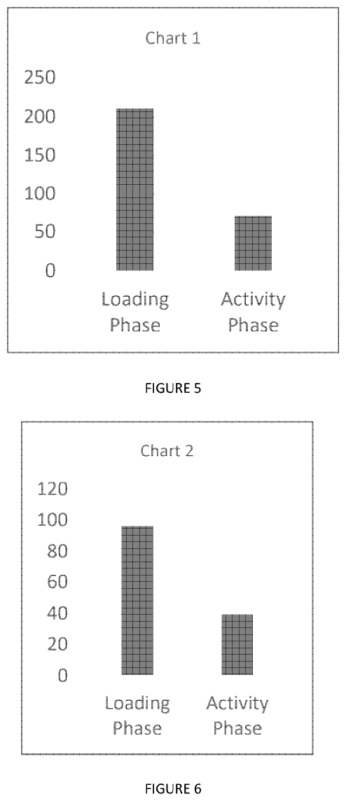

FIG. 6 is a graph illustrating the effect of the ectoparasiticide veterinary composition of the present invention on leg kicks of calves during the loading and activity phases;

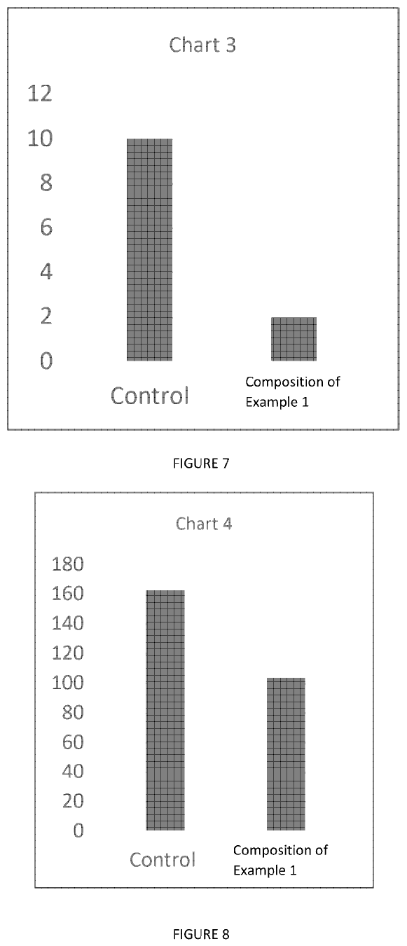

FIG. 7 is a graph illustrating the effect of the ectoparasiticide veterinary composition of the present invention on head turns of calves during the activity phase during the activity phase compared to the untreated sample; and

FIG. 8 is a graph illustrating the effect of the ectoparasiticide veterinary composition of the present invention on tail swishing of calves during the activity phase during the activity phase compared to the untreated sample.

### DETAILED DESCRIPTION

### Example 1—Ectoparasiticide Veterinary Composition

The composition comprises:

54.25% diallyl sulphide (DASn) where n is equal to or greater than 4 (further comprising DASn where n is equal to or greater than 5, further comprising DASn where n is equal to or greater than 6)

14% alliclove liquid

8% Chelated zinc

9% Methionine liquid

6% Seaweed concentrate

5.5% Brinex Botanical

3% MSM

0.75% Liquorice Powder/Crystal

0.5% Processing aids

### Example 2—Red Mite Infestation

Red mite (Dermanyssus gallinae) is an ectoparasite commonly found in poultry houses. Red mite are nocturnal blood feeding ectoparasites and affect 72% of poultry in Turkey. The average contact between the red mite and the chicken normally occurs over a 1-2 hour period and due to their size the ectoparasites are barely visible.

The red mites reach adulthood within 7 days and will lay over 30 eggs on the host animal. Therefore the red mite infestation on the host animal can multiply rapidly. The lifespan of a mite is between 6 to 9 months and have a direct impact on the reduction in profitability of the host chicken (estimated to be between 7-10% reduction per chicken).

It has been found that a red mite infestation can result in:
10-15% decrease in egg production;
up to 2.2% decrease in egg weight;
decrease in egg quality due to bark thinning and staining
up to 5.7% decrease in feed efficiency;
decrease in live weight;
stress, aggression and death in hens.

Red mites are blood feeding ectoparasites, and as such there may be associated losses due to the transmission of diseases via the blood system between chickens or from the ectoparasite to the chicken.

Chemical treatments have resulted in a rise in antimicrobial resistance of the red mites. As a result, many, once effective, chemical treatments have been found to have little impact on red mite infestations. Increased use of chemical treatments to treat ectoparasite infestations, such as red mites, has exacerbated the problem. In 2004, a study conducted in England found that more than 60% of poultry houses were found to have a resistance to conventional chemical treatments. There is also a risk that residues of the chemicals or drugs used in the treatments may remain in the meat or be transmitted to the eggs. In some cases, 91% of laying chickens were found to contain dangerous levels of drug residues.

The ectoparasiticide veterinary composition of Example 1 is water-soluble and is administered to chickens via the in-line water system.

Once administered, the ectoparasiticide veterinary composition of Example 1 is absorbed through the gut wall and into the vascular system of the chicken. The synergy of the ectoparasiticide veterinary composition of Example 1 allows the delivery of the compounds of Formula (I) and essential oils to the dermis and epidermis of the chicken where the Compound of Formula (I) stimulates an adverse reaction through the sensory receptors of the red mites and as such creates a repellent effect.

The animal feed comprising ectoparasiticide veterinary composition of Example 1 was trialled on two properties in Izmir, Turkey having known red mite infestations. Farm 1 housed 80000 layers and Farm 2 housed 30000 layers. The animal feed comprising the ectoparasiticide veterinary composition of Example 1 was introduced on Day 1 and observations were made daily.

No impact or reduction was noted between day 1 and day 14. On day 15, it was noted that mites appeared to be less prevalent. Between day 16 and day 20 the reduction in infestation continued to be seen. By day 21, it was established that the use of the animal feed comprising ectoparasiticide veterinary composition of Example 1 of the present invention through the water system had a significant impact on the reduction of red mite infestation within the poultry houses.

### Example 3—Red Mite Infestation

Two broiler houses of poultry were used in the trial, a first “trial house” and a second “control house”. Mortality was recorded weekly to determine the red mite impact on the poultry. Death within poultry is typically a result of anaemia or disease caused by secondary infection.

In the trial house, the ectoparasiticide veterinary composition of Example 1 was administered to chickens via the in-line water system at a dose of 4 ml per 10 litres of clean water. The control house used the farm's standard medication program involving the administration of antibiotics (Fosfomycin and enrofloxacin). Flock performance monitoring (measuring the number of mortalities not including the standard cull numbers) was recorded weekly in each house over a 28 day period. The mortality results over the 28 day period are shown in FIG. 1. FIG. 2 shows the improvement (%) of mortality in the trial house compared to the control house each week over the 28 day period. It can be seen from FIG. 1 that the mortality % for the control house increased to 5% over the 28 day period. In contrast, the mortality % for the trial house was significantly lower (3.5%) over the 28 day period.

FIG. 2 shows that over the 28 day period, the trial house provided a 41% improvement (i.e. reduction) in mortality amongst the poultry compared to the control house.

### Example 4—the Effect of Red Mite Infestation on Broiler Birds

Two poultry houses were used in the trial: a “trial house” and a “control house”. Each house contained 37000 broiler birds.

In the trial house, the ectoparasiticide veterinary composition of Example 1 was administered each afternoon continuously for 6 hours at a dosage of 1 ml per 1 litre of clean drinking water. The control house used the farm's standard medication program involving the administration of antibiotics (Fosfomycin and enrofloxacin).

Average live weight of the birds in each house was recorded weekly for a 28 day period. At the end of the trial a feed conversion ration was identified.

FIG. 3 shows the weekly average live weight in each of the houses. In the control house, the final average live weight (at 28 days) was recorded as 1370 g. In contrast, in the trial house, the final average live weight (at 28 days) was recorded as 1660 g. This shows that the ectoparasiticide veterinary composition of the present invention achieved an average live weight gain which was 21.17% higher than the average live weight gain of broiler birds treated with antibiotics.

The feed conversion ratio had an improvement of 18% in the trial house.

### Example 5—Buffalo Fly Infestation

The animal feed comprising ectoparasiticide veterinary composition of Example 1 of the present invention was used to control the impact of Buffalo Fly (Haematobia irritans exigua) on cattle. The animal feed was introduced in liquid form to a freely available supplementary cattle feed consisting of molasses and rations of grains, Rumensin and trace elements. Mixing occurred mechanically using an engine driven paddle mixer to ensure all ingredients are thoroughly blended to an even consistency. The ectoparasiticide veterinary composition of Example 1 was added to animal feed to be administered to the host animal at a target dose rate of 5 mg per head per day.

The trial involved 146 head of weaner cattle located in Northern Australia. Cattle have access to the supplementary feed at all times.

The trial commenced when Buffalo Fly numbers were low and starting to increase in line with the warmer weather and were clearly visible on cattle. As fly numbers were initially low, there were little to no visible animal impact signs on the cattle. At week four, fly numbers had increased significantly and were clearly visible on the cattle. Regular inspections were made and the presence of medium numbers of flies on most cattle was noted however only four animals within the herd showed any visible signs of impact (i.e. small lesions around the eyes).

The herd was compared to a herd on a neighbouring property which had not been administered with the animal feed of the present invention (i.e. a control herd). The control herd was found to have significant lesions evident around the eyes and neck of the cattle. The ectoparasiticide veterinary composition of Example 1 was found to have reduced the impact of Buffalo flies to a low-medium level of infestations.

The ectoparasiticide veterinary composition of Example 1 of the present invention was therefore demonstrated to be effective in preventing the buffalo flies from biting and feeding on the cattle. The ectoparasiticide veterinary composition of Example 1 did not in this instance prevent the flies from landing on the cattle, however the ectoparasiticide veterinary composition of Example 1 did stop the flies from biting the animals and therefore substantially reduced irritation and subsequent development of skin lesions at the site of irritation. Furthermore, the ectoparasiticide veterinary composition of Example 1 provided the additional advantage of preventing transmission of parasitic worms which can infest fly bites and result in the development of open sores which require additional treatment and can detrimentally effect the quality of the hide. The ectoparasiticide veterinary composition of Example 1 was also found to not have any negative effect on the dung beetle population.

The ectoparasiticide veterinary composition of Example 1 of the present invention has the benefit of:
an almost complete absence of Buffalo Fly bites and resultant lesions in cattle;
maintenance of live weight gain;
use of all natural ingredients;
no costs and handling of chemicals to treat Buffalo Fly infestations;
not having to muster and yard cattle to administer chemical treatments resulting in calmer, less stressed cattle environment and reduced handling time;
not having to install and maintain back rubbers to control fly numbers;
no risk of developing chemical resistance to chemical treatments;
ease of delivery to cattle and incorporation into existing feeding program;
not having to maintain withholding periods if animals are sold for slaughter due to risk of residual drugs or chemicals within or on cattle;
not requiring a withdrawal period prior to slaughter.

A key benefit to the farm is not having to provide a withdrawal period prior to slaughter due to the natural agents present within the composition. It is known that the last couple of weeks, prior to slaughter, are essential in order to achieve good final slaughter weights, therefore it is advantageous to be able to protect the host animal during this time.

### Example 6—Treatment of Flies, Ticks and Mosquitoes on Horses

The ectoparasiticide veterinary composition of Example 1 was premixed onto a calcium silicate at 50% and then added to the horse feed Goldhorse at 0.2%. This was then fed a minimum of 2.5 kg of the Goldhorse a day equating to 2.5 g per day of the composition of Example 1.

The study extended to 200 horses over 9 stables.

After 2 days of administration, the ticks had disappeared from the horses. The ectoparasiticide veterinary composition of Example 1 was found to have 100% success rate at keeping or repelling ticks away from the horses.

After 2 weeks of administration, the number of flies on the horses in a stable environment had reduced by 50-60%. The same administration process was carried out in a higher fly stress environment, i.e. on horses within a swamp-style environment. After 2 weeks of administration, the same 50-60% reduction in fly numbers was observed.

After 2 weeks of administration, mosquito nuisance and bites had disappeared from 199 of the 20 horses within the study.

It can be seen that the ectoparasiticide veterinary composition of Example 1 of the present invention successfully reduced and repelled the ectoparasites, including ticks, flies and mosquitos, from the host animal.

### Example 7—Effect of Ectoparasiticide Veterinary Composition on Ticks on Calves

Two groups of calves (a trial group and a control group) were fed a standard diet which included a feed pellet. In the trial group, the feed pellet was pre-treated with the ectoparasiticide veterinary composition of Example 1 at a dosage of 4 g per head per day administered over two feeds.

Efficacy was determined by a forced tick infestation challenge. Ticks were placed inside a tick feeding patch which was created by gluing a stockinet to a short-clipped ring on the back of the calves. Inside the stockinet enclosure a 25 cm diameter ring of normal hair length was maintained and ticks were placed onto the hair during the infestation process. Patches were removed after 24 hours and ticks (attached and unattached) were counted.

A pre-trial challenge was carried out on Day 0 on all animals. No significant difference was determined between the two groups of calves.

The host animals were not challenged during the 21 day loading phase. Activity of the host animals began at day 22. At day 24, a second challenge was completed.

The results are shown in FIG. 4 where it can be seen that the trial group demonstrated a 43% reduction in tick attachment to the host animal compared to the control group.

### Example 8—Effect of Ectoparasiticide Veterinary Composition on Egg Staining

A first group of poultry was fed with on water treated with the ectoparasiticide veterinary composition of Example 1. A second group of poultry was fed on untreated water (control sample).

Ten eggs from each poultry group were hard-boiled in separate pans for 10 minutes in simmering hot water. After cooling and shelling, the eggs were processed in two blenders for 20 seconds on speed 2 until finely chopped. The egg samples were presented to a panel of 18 assessors at ambient temperature.

The samples were evaluated using the Triangle Test Procedure (TES-S-001). In the triangle test each assessor is presented with a set of three coded samples, two of which are the same, and one which is different. The sets of samples are presented equally often in each of the six possible orders. This experimental design minimises any possible order and carry over effects.

Eighteen trained assessors were used for each test, nine receiving “treated eggs” as the “different” sample and nine receiving “control eggs” as the “different sample”. After tasting the samples, in the designated order, each assessor is asked to select the different sample and to describe the difference perceived.

The test was carried out in a testing room which is positively pressurised to minimise the entrance of external odours. Orange colour lighting was used to mask the colour difference between the samples.

Out of 18 assessors, 10 correctly identified the differing sample.

There was a statistically significant difference detected between the two samples of eggs at 5% level of significance. It was however noted that no taint descriptors were used by the panel for either egg sample.

It is therefore concluded that the ectoparasiticide veterinary composition of Example 1 does not taint eggs produced by treated poultry.

### Example 9—Stability of Ectoparasiticide Veterinary Composition

Garlic extracts such as garlic oil are volatile substances and the pharmacologic actions of these extracts are dependent on ratio of the elements present within.

The ectoparasiticide veterinary composition of the present invention comprises the most efficient combination of polysulphide compositions and other components. The stability of the volatile substances is controlled and stabilised by the presence of acetic acid.

Stability of the ectoparasiticide veterinary composition of Example 1 in a non-Newtonian liquid were tested under extreme short term conditions of 54% for 2 weeks and 7 days storage at OC. The ectoparasiticide veterinary composition of Example 1 was found to be stable within these parameters with no separation of the liquid phase.

The ectoparasiticide veterinary composition of the present invention were found to have good dispersibility and homogeneity and excellent suspensibility and did not appear to separate in short and medium term testing.

After a year at 20C the ectoparasiticide veterinary composition of Example 1 appeared to be completely stable with no evidence of separation or sedimentation or loss of activity.

### Example 10—Comparison Doses for Treatment of Other Animals

The ectoparasiticide veterinary composition of the present invention can be administered to a wide variety of animals. The dosage required depends on the type of animal to be treated for ectoparasitic infestations.

For example, the ectoparasiticide veterinary composition of the present invention may be administered to cattle and deer at a dosage of 5-6 grams of composition per head per day. In order to treat or reduce tick nuisance within sheep, the ectoparasiticide veterinary composition of the present invention may be administered at a dosage of 3 grams per head per day. ectoparasiticide veterinary composition of the present invention may be administered to equines at a dosage of 5 grams per head per day.

With regards to the treatment of ectoparasites on dogs, the ectoparasiticide veterinary composition of the present invention may be administered at different dosages depending on the size or breed of the dog. The ectoparasiticide veterinary composition of the present invention may be mixed into the dog food or the water. The dosages may be: for a large breed dog—1 gram of composition per head per day; for a medium breed dog—0.6 grams of composition per head per day; and for a small breed dog—0.3 grams of composition per head per day.

For the treatment of ectoparasites on cats, the ectoparasiticide veterinary composition of the present invention may be administered by mixing into the cat feed at a dosage of 0.2 grams of composition per head per day.

### Example 11—Method of Preparation of Ectoparasticide Composition

The garlic cloves are placed in acetic acid (preferably cidar apple vinegar). The pH is maintained between 4.5 and 6.0. The solution is heated to an optimum temperature of 35° C. The garlic cloves are chopped whilst present within the acetic acid to provide the highly reactive precursor of allicin, 2-propenesulphenic acid. 2-propenesulphenic acid dimerizes to form allicin. After 24 hours, the solution comprises DASn where n is at least 4, preferably including DAS7-22, making this a unique source DASn where n is at least 4.

### Example 12—Treatment of Sparicotyle chrysophrii on Fish

The effect of the ectoparasiticide veterinary composition of the present invention was investigated on four different breeds of fish: Sea Bass, Amber Jack, Sea Bream and Red Sea Bream.

A control sample of fish was fed fish food untreated with the ectoparasiticide veterinary composition of the present invention.

Treated samples of fish were administered with the ectoparasiticide veterinary composition of the present invention sprayed onto the fish feed.

For young fish (with a weight in the range of between 2 g and 200 g), the ectoparasiticide veterinary composition of the present invention was sprayed onto the fish feed at a dosage of 1 litre of ectoparasiticide veterinary composition of the present invention per 1000 kg of fish feed. The young fish were pulse fed under a regime where the ectoparasiticide veterinary composition of the present invention was administered over alternate 7 day periods.

For mature fish (with a weight in the range of between 200 g and 500 g), the ectoparasiticide veterinary composition of the present invention was sprayed onto the fish feed at a dosage of 0.5 litre of ectoparasiticide veterinary composition of the present invention per 1000 kg of fish feed. The mature fish were fed under a regime where the ectoparasiticide veterinary composition of the present invention was administered for 7 days at the start of each month.

The results showed that the treated fish samples had a reduced mortality rate compared to the control samples of fish. The mortality rate of treated fish was reduced by 32% compared to the mortality rate of control fish samples.

The results also showed that the oxygen levels within the gills of the treated fish samples were higher when compared to the control samples of fish.

The results also showed that the presence of the ectoparasite Sparicotyle chrysophrii had been eradicated within the treated fish samples (but was still present within the control fish samples).

Although this study investigated the treatment of the ectoparasite Sparicotyle chrysophrii it is to be understood that the effect of the ectoparasiticide veterinary composition of the present invention is not limited to this specific ectoparasite and can be used to treat and eradicate all ectoparasite infestations on a host animal.

The results showed that the treated fish samples had reduced levels of parasitic invasion compared to control fish samples.

The results also showed that the treated fish samples had a greater weight gain of 70 g to 80 g over 35 days compared to the weight gain of 30 g to 40 g over 35 days of the control fish sample. It was also found that the feed conversion ratio (i.e. the amount of food required to be digested in order to provide a predetermined weight gain within the host animal) was improved (i.e. less feed required in order to provide the predetermined weight gain) for feed treated with the ectoparasiticide veterinary composition of the present invention. In particular, the control fish sample required 2.50 g of fish feed to provide a 1 g fish live weight. In contrast, the treated fish sample required 1.83 g of fish feed to provide 1 g fish live weight. As a result, it can be seen that the feed conversion of animal feed treated with the ectoparasiticide composition of the present invention was far more efficient compared to untreated animal feed. This demonstrates that the ectoparasiticide composition of the present invention achieves a 37% increase in feed efficiency which significantly reduces the associated feed costs.

The results also showed that the treated fish samples display higher levels of immunoglobulins compared to control fish samples.

### Example 13—Effect of Ectoparasiticide Veterinary Composition on Animal Behaviour

The effect of the ectoparasiticide veterinary composition of the present invention was investigated on the animal behaviour of calves.

A first sample (a control sample) of calves was fed animal feed in the form of feed pellets untreated with the ectoparasiticide veterinary composition.

A second sample (a treated sample) of calves was fed animal feed in the form of feed pellets treated with the ectoparasiticide veterinary composition.

The treated feed pellets were fed to the calves at a dosage rate of providing 4 g per head per day of the ectoparasiticide veterinary composition of the present invention.

Efficacy of the ectoparasiticide veterinary composition was determined by monitoring recognised animal behavioural characteristics faced with an introduced fly challenge. These animal behavioural characteristics are: tail swishing, head turns, and leg kicks.

The control sample and the treated sample were monitored both during the loading phase (i.e. during the 21 days of feeding) and the activity phase (105 days; late June to early October) when the ectoparasiticide veterinary composition is considered to have reached peak effectiveness.

With reference to FIGS. 4 to 7, it can be seen that there was a significant behavioural difference observed in animals treated with ectoparasiticide veterinary composition of the present invention compared to untreated animals. It can be seen that distress and irritation caused by fly nuisance can be reduced by an average of 60% when fed the ectoparasiticide veterinary composition of the present invention compared to untreated host animals. As a result, it can be concluded that the ectoparasiticide veterinary composition of the present invention can be used to reduce negative and/or stressed behaviour associated with ectoparasite infestations.

### Example 14—Effect of Ectoparasiticide Veterinary Composition on Bacteria

An ectoparasiticide veterinary composition of Example 1 (labelled here as PST22) was tested in an in vitro experimental laboratory to test its efficacy against conventional antibiotics used to control harmful bacteria and fungus that cause untold harm in modern poultry producers. Leading world authorities including the World Health Organisation (WHO) have graded Antimicrobial Resistance (AMR) as a serious global health issue that threatens the prevention and treatment of an increasing range of infections.

ESKAPE is an acronym that encompasses the scientific names of six bacterial pathogens commonly associated with AMR and of clinical significance to the medical and agricultural field. These pathogens are: Enterococcus faecium; Staphylococcus aureus; Klebsiella pneumoniae; Acinetobacter baumannii; Pseudomonas aeruginosa; and Enterobacter spp. (ESBLs and Carbapenemases).

The results are shown in Tables 5 to 8.

TABLE 5

| | | | | | +++ = best (−) = worst |
|---|---|---|---|---|---|
| | | | | | Inhibitory Activity |
| | | | | Average of 3× trials | (stopping power |
| | Escherichia coli (ATCC25322) | | | Reactivity Killing | thoroughness and |
| Sample/Control | Trial 1 | Trial 2 | Trial 3 | Power (mm) | lasting effect) |
| A PST22 | 18.28 | 16.16 | 17.41 | 17.28 | +++ |
| (Undiluted) (10 mm) | | | | | |
| B PST 22 | 10.00 | 10.00 | 10.00 | 10.00 | +++ |
| (Diluted by 1000 times) | | | | | |
| Positive Control | 16.20 | 16.39 | 16.12 | 16.24 | +++ |
| Amikacin 30 ug (6 mm) | | | | | |
| Negative Control | 0.00 | 0.00 | 0.00 | 0.00 | (−) |
| Sample-free disc (10 mm) | | | | | |

Table 5 shows that the ectoparasiticide veterinary composition of Example 1 outperformed the current chosen antibiotic by 6.4% for killing E-Coli when undiluted. When diluted by 1000 times, the ectoparasiticide veterinary composition of Example 1 was still found to have very powerful inhibitory activity, similar to the antibiotic (positive control).

TABLE 6

| | | | | | +++ = best (−) = worst |
|---|---|---|---|---|---|
| | | | | | Inhibitory Activity |
| | | | | Average of 3× trials | (stopping power |
| | Staphylococcus aureus (ATCC 6538P) | | | Reactivity Killing | thoroughness and |
| Sample/Control | Trial 1 | Trial 2 | Trial 3 | Power (mm) | lasting effect) |
| A PST22 | 22.53 | 20.26 | 23.31 | 22.03 | +++ |
| (Undiluted) (10 mm) | | | | | |
| B PST22 | 0.00 | 0.00 | 0.00 | 0.00 | (−) |
| (Diluted by 1000 times) | | | | | |
| Positive Control | 22.82 | 20.20 | 20.72 | 21.25 | +++ |
| Oxacillin 1 ug (6 mm) | | | | | |
| Negative Control | 0.00 | 0.00 | 0.00 | 0.00 | (−) |
| Sample-free disc (10 mm) | | | | | |

Table 6 shows that the ectoparasiticide veterinary composition of Example 1 outperformed the current chosen antibiotic by 3.7% for killing Staphylococcus aureus. This harmful bacteria is on the ESKAPE WHO list. Therefore the composition of the present invention may be used effectively to treat bacterial diseases without any risk of antibiotic resistance.

TABLE 7

| | | | | | +++ = best (−) = worst |
|---|---|---|---|---|---|
| | | | | | Inhibitory Activity |
| | | | | Average of 3× trials | (stopping power |
| | Salmonella typhimurium (ATCC 14028) | | | Reactivity Killing | thoroughness and |
| Sample/Control | Trial 1 | Trial 2 | Trial 3 | Power (mm) | lasting effect) |
| A PST22 | 14.83 | 16.40 | 14.92 | 15.38 | +++ |
| (Undiluted) (10 mm) | | | | | |
| B PST22 | 10.00 | 10.00 | 10.00 | 10.00 | +++ |
| (Diluted by 1000 times) | | | | | |
| Positive Control | 16.12 | 16.26 | 16.52 | 16.30 | +++ |
| Amikacin 30 ug (8 mm) | | | | | |
| Negative Control | 0.00 | 0.00 | 0.00 | 0.00 | (−) |
| Sample-free disc (10 mm) | | | | | |

Table 7 shows that the ectoparasiticide veterinary composition of Example 1 performed with similar activity to the current chosen antibiotic. When diluted 1000 fold, the composition of Example 1 had 61% of the antibacterial activity of the chosen antibiotic with equal inhibitory activity.

TABLE 8

| | | | | | +++ = best (−) = worst |
|---|---|---|---|---|---|
| | | | | | Inhibitory Activity |
| | | | | Average of 3× trials | (stopping power |
| | Pseudomonas aeruginosa (ATCC 27853) | | | Reactivity Killing | thoroughness and |
| Sample/Control | Trial 1 | Trial 2 | Trial 3 | Power (mm) | lasting effect) |
| A PST22 | 17.53 | 16.63 | 16.54 | 16.90 | +++ |
| (Undiluted) (10 mm) | | | | | |
| B PST22 | 10.00 | 10.00 | 10.00 | 10.00 | +++ |
| (Diluted by 1000 times) | | | | | |
| Positive Control | 17.89 | 17.08 | 17.02 | 17.33 | +++ |
| Amikacin 30 ug (8 mm) | | | | | |
| Negative Control | 0.00 | 0.00 | 0.00 | 0.00 | (−) |
| Sample-free disc (10 mm) | | | | | |

Table 8 shows that the composition of Example 1 performed as well as the current chosen antibiotic against Pseudomonas aeruginosa. When diluted 1000-fold, the composition of Example 1 had 57% of PG-6T the activity of the antibiotic and equal inhibitory activity. Again, Pseudomonas aeruginosa is on the ESKAPE WHO list. The composition of the present invention may be used to effectively treat bacterial diseases without any risk of antibiotic resistance developing.

### Example 15—Effect of Ectoarasiticide Veterinary Composition on Fungi

An ectoparasiticide veterinary composition of Example 1 (labelled here as PST22) was tested in an in vitro experimental laboratory to test its efficacy against conventional antibiotics used to control harmful bacteria and fungus that cause untold harm in modern poultry producers.

TABLE 9

| | Saccharomyces cerevisiae | | | | Average of | +++ = best |
|---|---|---|---|---|---|---|
| | (ATCC 9763) | | | | 3× trials | (−) = worst |
| | | | | Total Mean | Reactivity | Inhibitory Activity |
| | | | | Zone of | Killing | (stopping power |
| | | | | Inhibition | Power | thoroughness and |
| Sample/Control | Trial 1 | Trail 2 | Trial 3 | (mm) | (mm) | lasting effect) |
| PST22 - B | 0.00 | 0.00 | 0.00 | 0.00 | 0 | (−) |
| (1 ml/L) (10 mm) | | | | | | |
| Positive Control Nystatin | 21.28 | 21.30 | 22.17 | 21.58 | 4 | +++ |
| (10 mm) | | | | | | |
| Negative Control Sample Free | 0.00 | 0.00 | 0.00 | 0.00 | 0 | (−) |
| (10 mm) | | | | | | |

Saccharomyces cerevisiae is a live yeast that is commonly used in poultry diet for improving digestion.

Table 9 shows that the composition of Example 1 may be administered to host animals without adversely affecting live yeasts which are present for improved digestion. As such, the composition of Example 1 is compatible with all know pre and pro-biotics.

TABLE 10

| | Aspergillus niger (ITDI Ref. No. 3008) | | | | +++ = best (−) = worst |
|---|---|---|---|---|---|
| | | | | Average of 3× trials | Inhibitory Activity |
| | | | | (larger the better) | (stopping power |
| | | | | Reactivity killing | thoroughness and |
| Sample/Control | Trial 1 | Trial 2 | Trial 3 | power (mm) | lasting effect) |
| PST22 - A | 27.76 | 26.04 | 25.20 | 26.33 | +++ |
| (pure) (10 mm) | | | | | |
| PST22 - B | 10.00 | 10.00 | 10.00 | 10.00 | +++ |
| (1 ml/L) (10 mm) | | | | | |
| Positive Control | 15.80 | 16.20 | 16.18 | 16.06 | +++ |
| Clotrimazole (10 mm) | | | | | |
| Negative Control | 0.00 | 0.00 | 0.00 | 0.00 | (−) |
| Sample Free (10 mm) | | | | | |

Table 10 shows that the composition of Example 1, is highly effective in controlling fungi, in particular Aspergillus niger, in the feed or in the environment which, if left unchanged, can cause respiratory, nervous and eye problems in host animals such as chickens. Table 10 shows that the composition of Example 1 outperforms Clotrimazole by 64% when undiluted. When diluted by 1000-fold, the composition of the present invention displays very powerful inhibitory activity equal to the antibiotic.

1. An ectoparasiticide composition comprising;
an extract of Allium sativum comprising at least one compound of the formula (I): 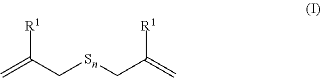
wherein n is at least 8 and in which R¹ is selected from the group consisting of: hydrogen; halogens; substituted or unsubstituted C₁₋₅ alkyl groups;
substituted or unsubstituted phenyl groups; carboxy group; carboalkoxy groups;
hydroxymethyl; and trimethylsilylmethyl.

2. The ectoparasiticide composition of claim 1, in which R¹ is selected from the group consisting of: hydrogen;
chloro; fluoro; methyl; C₂₋₄ alkyl groups; phenyl; carboxy;
carbomethoxy; carboethoxy; hydroxymethyl; and trimethylsilylmethyl.

3. The ectoparasiticide composition of claim 1, in which the composition comprises at least one compound of the formula (I) in which n is between 8 and 22.

4. The ectoparasiticide composition of claim 1, further comprising acetic acid.

5. The ectoparasiticide composition of claim 1, in which the pH of the composition is between 4 and 6.

6. The ectoparasiticide composition as claimed in claim 5, in which the pH of the composition is between 4.1 to 5.9.

7. The ectoparasiticide composition as claimed in claim 6, in which the pH of the composition is between 4.2 and 4.9.

8. The ectoparasiticide composition of claim 1, in which the composition is substantially free from allicil.

9. A The ectoparasiticide of claim 1, further comprising one or more of: allyl methyl disulphide, dimethyl trisulphide, allyl-1-propenyl disulphide, allyl-1-propenyl trisulphide, 2-vinyl-4H-1,3-dithine, methyl-2-propenyl thiosulphinate.

10. The ectoparasiticide composition of claim 1, further comprising one or more of: methiin, alliin, and/or propiin, and/or the thiosulphinates thereof.

11. The ectoparasiticide composition of claim 1, further comprising one or more of:
sulphur-containing precursor compounds for allicin; and/or
an extract of Seaweed; and/or
methionine; or
any combination thereof.

12. The ectoparasiticide composition as claimed in claim 11, in which the sulphur-containing precursor compounds for allicil comprise 2-propenesulphenic acid.

13. An animal feed composition comprising an ectoparasiticide composition of claim 1.

14. The animal feed composition as claimed in claim 13, in which the animal feed composition is a terrestrial and/or an aquatic animal feed composition.

15. The animal feed composition of claim 13, in which the composition is administered in the animal drinking water.

16. A method comprising:
administering to an animal the Use of an ectoparasiticide composition of claim 1 in a treating, in a controlling or in a reducing a likelihood of ectoparasitic infestations effective amount.

17. The method of claim 16, wherein the ectoparasiticide composition is part of an animal fee composition.

18. The ectoparasiticide composition of claim 2, in which the composition comprises at least one compound of the formula (I) in which n is between 8 and 22.

19. The ectoparasiticide composition of claim 2, further comprising acetic acid.

20. The ectoparasiticide composition of claim 2, in which the pH of the composition is between 4 and 6.
